(19) United States
(12) Patent Application Publication
(10) Pub. No.: US 2022/0054031 A1
(43) Pub. Date: Feb. 24, 2022

(54) A METHOD FOR CALIBRATING A BLOOD PRESSURE MONITOR, AND A WEARABLE DEVICE THEREOF

(71) Applicant: WELL BEING DIGITAL LIMITED, New Territories (HK)

(72) Inventor: Ming Yip Wallace WONG, New Territories (HK)

(21) Appl. No.: 17/413,922

(22) PCT Filed: Oct. 29, 2019
(86) PCT No.: PCT/CN2019/113914

§ 371 Date: Jun. 14, 2021

(30) Foreign Application Priority Data
Dec. 13, 2018 (HK) 18116042.4

(51) Int. Cl. A61B 5/024; A61B 5/022; A61B 5/00

(52) U.S. Cl. CPC A61B 5/02416; A61B 5/02241; A61B 2562/0219; A61B 2560/0223; A61B 5/6817

(57) ABSTRACT

Methods and related devices 100 for obtaining the blood pressure of a person in two different ways. The first way comprising steps of providing a light source 101, and an optical sensor 103 configured to detect light from the light source 101 which has propagated through the wrist of a wearer of such a device 100. The amount of light propagating through the wrist depends on the amount of blood in the wrist. Blood pressure is then observed by monitoring the difference in the amplitude of blood pulsation within the wrist when the wrist is lifted above heart level, and when the wrist is lowered below heart level. The second way comprising mathematically analysing light transmission signals though a body part of the same person. Blood pressure readings obtained by the first way are used to calibrate blood pressure readings by the second way.

### FIELD OF INVENTION

This invention relates to calibration of an apparatus or device for monitoring blood pressure (BP), and particularly to calibration of wearable blood pressure monitors.

### BACKGROUND OF INVENTION

Blood pressure is measured as part of routine and basic medical examination. Knowing the blood pressure of a person, or a subject, aids medical diagnosis and a medical practitioner may be alerted to hidden ailments if the blood pressure of a person is too high or too low.

Blood pressure is fully measured when both systolic blood pressure and diastolic blood pressure are measured. Systolic blood pressure refers to pressure in blood vessels when the heart contracts while diastolic blood pressure refers to pressure in blood vessels when the heart is relaxed.

A device for measuring blood pressure is called a sphygmomanometer. There are manual and digital sphygmomanometers. A manual sphygmomanometer comprises a cuff to be applied around the bicep tightly, positioned at roughly the same level as the heart while the person is seated. The cuff is tightened to strangle the bicep. The stranglehold is released slowly while the practitioner listens to the flow of pounding blood in the brachial artery at the elbow using a stethoscope. The cuff pressure beginning at which sound of pounding blood may be heard is recorded as the systolic blood pressure. The manual sphygmomanometer is installed with a mercury column to allow the operator to read the cuff pressure. As the cuff pressure is released further, the cuff pressure beginning at which the sound of pounding blood can no longer be heard is recorded as the diastolic blood pressure.

While this method of taking blood pressure is generally accurate, inaccuracies do arise from operator error, poor use of equipment, and inadequate equipment maintenance.

The digital sphygmomanometer was developed for portability, and for obviating the need to use mercury and the need for operator training. While lacking the fine accuracy of a mercury column, the digital sphygmomanometer has been found accurate enough for use in a domestic environment by the user himself. Most digital sphygmomanometers require a pressurised cuff to be tightened around the bicep of the user in the same way as a manual sphygmomanometer. Some digital sphygmomanometers require only the cuffing of the wrist or a finger. In any case, the part which is cuffed must be positioned about the same level as the heart. Both the systolic and diastolic pressures are sensed indirectly using piezoelectric, capacitive or electrostatic pressure sensors. The actual blood pressure is calculated by comparing pressure readings against calibration. Despite the perceived ease of use, a somewhat elaborate routine is required for the person to put on the cuff in order to allow the digital sphygmomanometers to operate. It is often inconvenient for a person who needs to have his blood pressure level measured rather urgently in a crowded urban location.

Xing et al. proposed to obtain blood pressure measurement of people by using a photoplethysmogram to observe blood volume changes, and calculating blood pressure from the volume change (Vol. 7, No. 8|1 Aug. 2016|BIOMEDICAL OPTICS EXPRESS 3007; Optical blood pressure estimation with photoplethysmography and FFT-based neural networks; XIAOMAN XING* AND MINGSHAN SUN). A photoplethysmogram is simply a spectrometric device measuring blood content and, hence, the pulse, using light transmission through a body part.

Typically, it is difficult to use light transmission to obtain objective evaluation of blood pressure. This is because of variation in blood vessel elasticity and blood volume between individuals. However, Xing et al. propose a method of normalizing PPG readings in order to remove these variations from observations. After normalization, the PPG signals become comparable between different persons. Accordingly, it is possible to make objective measurement of blood pressure using PPG alone.

FIG. 1 is a graph reproduced from a paper published by Xing et al., showing how PPG signal is normalized. The graph on the left shows a person's pulse, or heartbeat, as an alternating current (AC) component riding on an underlying direct current (DC) component. On normalization, the DC component is removed, as shown by the graph on the right. The process of normalization is performed by dividing the AC part by the DC part, and the resultant signal is scaled linearly to provide PPG readings within a certain amplitude range

Xing et al. proposes a formula for correlating the PPG signal to blood pressure, by which the scaled PPG signal is related to the normalized PPG signal.

PPG_{scaled}=kₛ×PPGₙₒᵣₘ+V_{off}≈k(e^{-γP}^{ₘᵢₙ}−e^{-γP})+V_{off} (1)

where
PPGₙₒᵣₘ is the normalized PPG signal as illustrated n FIG. 1
P is transmural pressure, across the arterial wall
γ is a constant which depends on the measurement site and the animal of which blood pressure is being measured, for example, in canine aorta γ is 0.017±0.0004 mm Hg⁻¹
k is a linear scaling factor, which is used to amplify and shift the normalized PPG signal, and may vary from manufacturer to manufacturer
V_{off} is an offset factor

Xing et al. assumed that the PPG signal correlates linearly with the blood volume V in the body part on which measurement is performed.

For completeness, the mathematical deliberation of Xing et al. is reproduced herein below. Basically, PPGₙₒᵣₘ is obtained from combining the following three basic equations

$$\begin{matrix} E = {E}_{0} {e}^{γ P} & ( 2 ) \\ E = ( 1 - {σ}^{2} ) \times \frac{{r}^{2}}{\text{?}} \times \frac{dB}{dr} & ( 3 ) \\ V = C π {r}^{2} + {V}_{0} \\ \text{?} \text{indicates text missing or illegible when filed} & ( 4 )\end{matrix}$$

Equation (2) was developed by Hughs et al. for blood vessels, and simply adapts the Young's modulus for determining the stiffness of material,
wherein
E is Young's modulus of elasticity which determines the stiffness of blood vessels;
P is transmural pressure, across the arterial wall as explained for Equation (1); and
γ is a constant.

Equation (3) was developed by Bergal et al. I and Peterson et al.,
wherein
σ is set to 0 here, and is the Poisson's ratio (Poisson's ratio is a measure of the Poisson effect, the phenomenon in which a material tends to expand in directions perpendicular to the direction of compression. σ is set to 0 in the present application because It is determined that E should just represent circumferential stress);
r is the mean radius of the blood vessels;
h is the thickness of the blood vessel wall; and
P is transmural pressure as explained for Equation (1).

Equation (4) calculates the volume of blood in the arterial blood vessels in the body part on which measurement is performed, on assumption that vessel radius is a constant, wherein
C is a constant relating to blood density;
V_{Q} the volume of venous and microvascular blood.

Combining (1) to (3) provides the following equation.

$$V = \frac{{C ( {E}_{\text{?}} γ \text{?} )}^{2} π}{{( b + {s}^{- γ B} )}^{2}} \\ \text{?} \text{indicates text missing or illegible when filed} \qquad ( 5 )$$

wherein
b is a constant introduced by integration, and is assumed to be independent of E_{Q}, γ and h.

From Equation (5), V is approximated to the first order as

$$V \approx \frac{{C ( {E}_{s} γ \text{?} )}^{2} π}{{b}^{2}} ( 1 - \frac{2}{b} {e}^{- yP} ) \\ \text{?} \text{indicates text missing or illegible when filed} \qquad ( 6 )$$

The normalized PPG signal, i.e. per FIG. 1, can be related to the volumetric change of arterial blood over a period time, i.e.

$${PPG}_{norm} = \frac{V - {V}_{min}}{{V}_{min}} \qquad ( 7 )$$

Change in arterial blood can by extension be related to Equation (6). Hence, the normalized PPG signal can be related to pressure as follows.

$${PPG}_{norm} = \frac{V - {V}_{min}}{{V}_{min}} = \frac{2 ( {e}^{- γ {P}_{min}} - {e}^{- γ P} )}{b - 2 {\text{?}}^{- γ {P}_{min}}} \\ \text{?} \text{indicates text missing or illegible when filed} \qquad ( 8 )$$

As the time variant component of V is typically only a few percentage of the stationary component, Xing et al. consider that this means that PPGₙₒᵣₘ is very small. A scaling function is then used to amplify the normalized PPG signal.

Xing et al. used k as the scaling factor, and V_{off} as the offset factor. As b the constant introduced by integration is deemed to be very large, a modified scaling factor k₂ is used to simply the calculation, which produces Equation (1), already shown above.

PPG_{scaled}=k₂×PPGₙₒᵣₘ+V_{off}≈k(e^{-γP}^{ₘᵢₙ}−e^{-γP})+V_{off} (1)

Based on Equation (1), Xing et al. trained an artificial neural network to determine the systolic blood pressure and diastolic blood pressure from PPGₙₒᵣₘ.

The specific mechanism and design of the artificial neural network may be varied according to the state of the art, as would be known to the skilled man and does not require elaboration here.

Xing et al. are not the only ones who have attempted to determine blood pressure solely from PPG signal. This popular pursuit by different research groups is due to a desire to develop a PPG based blood pressure monitor that may be applied around the clock on the person. In fact, the paper published by Xing et al. cites several prior methods by other research groups.

However, a major disadvantage of all such methods of determining blood pressure from PPG signal is a need for constant re-calibration. A wearable device worn over a long period of many days is subject to disturbance from user movements. Hence, the device has to be re-calibrated as often as the reading may have become inaccurate or may have drifted. However, it is not possible for a consumer to have the skills and tools to re-calibrate a wearable device by himself in a domestic setting. It is also inconvenient for the consumer to visit regularly a trained technician using a clinical sphygmomanometer to calibrate the wearable device. Hence, even if PPG signal can be used to monitor blood pressure as proposed by Xing et al, such PPG devices become unreliable after a period of use simply because of inaccessibility to re-calibration.

Accordingly, it is desirable to provide a device or method, or both, which could possibly provide a convenient way of re-calibrating a PPG based blood pressure monitoring device.

### SUMMARY OF INVENTION

A method for calibrating a blood pressure monitor comprising steps of: providing the blood pressure monitor, the blood pressure monitor being capable of measuring blood pressure in two different ways; and using the first way of measuring blood pressure to calibrate the second way of measuring blood pressure. Typically, the blood pressure monitor is wearable by the person whose blood pressure is monitored.

This provides the advantage of a combination of different blood pressure measurement methods in the same embodiment, so that the most suitable measurement may be used in a given condition. For example, methods which are disruptive because they draw the attention of the person, such as methods that require tightening of a cuff around the person's bicep, tend to be more accurate but cannot be used continuously over a long period of time. These methods can be used to calibrate other methods which are relatively less disruptive but are also less accurate. Hence, the more accurate but disruptive method can be used to obtain one discrete, reading of blood pressure to calibrate the readings of the less accurate but less disruptive method. The less disruptive method can then be used to silently monitor the person's blood pressure over a long period of time, and be re-calibrated whenever necessary by the person using the embodiment himself.

Preferably, the first way obtains blood pressure by a physical-reaction-based measurement of the person wearing the blood pressure monitor. ‘Physical-reaction-based methods’ refers to methods that derive blood pressure from a physical parameter, such as height of a raised limb, or the pressure inside a body part which is exerted against a counter-pressure. Such methods usually require the person to participate or to be aware of the measurement taking place. However, measuring such physically visible, tactile or tangible parameters is the more reliable, objective method of deducing blood pressure.

Preferably, the second way obtains blood pressure by a static measurement of the pulse of the person wearing the blood pressure monitor. ‘Static’ refers to methods of monitoring blood pressure which does not require the person to be in a specific position, make any movement or feel any pressure applied onto him, and this could include methods which uses light transmission or light absorption made through a body part of the person such as using a photoplethysmogram. In other words, the measurement is taken in the ‘background’. Furthermore, static methods may include analysing electrocardiograms for blood pressure, analysing pulse transit time, pulse shape and so on. As static methods maybe applied without the attention of the person monitored, these methods are suitable for long term, continuous blood pressure monitoring.

Optionally, the static measurement comprises the step of: transmitting light through a body part to monitor blood content.

Preferably, the physical-reaction-based measurement comprises the step of: measuring the height to which a body part is lifted which manifests an accompanying change in blood content in the body part, from which blood pressure may be deduced.

Alternatively, the physical-reaction-based measurement comprises the steps of: strangling the body part with a known counter-pressure; and measuring the pressure exerted by a body part against the counter-pressure.

In some cases, the body part is a finger of the subject; and the strangling is provided by a ring worn on the finger.

Preferably, both the ‘first way’ and the ‘second way’ are performed by using a same photoplethysmogram sensor on the same body part. This provides the possible advantage of a single device worn on a part of the body. In some embodiments, the body part is the ear canal.

Alternately, the ‘first way’ and the ‘second way’ are performed using different photoplethysmogram sensors on different, respective body parts. This provides the possibility that the more accurate measurement read from one part of the body may be used to calibrate measurements made on another part of the body, where that other part of the body is more suited to wearing a blood pressure monitor for long term, continuous blood pressure monitoring.

In a further aspect, the invention proposes a wearable blood pressure monitor, comprising: a photoplethysmogram device for observing blood content in a body part of a person wearing the blood pressure monitor; a measurement device for measuring physical-reaction in the body part; a processing device for interpreting the physical-reaction in the body part into a first blood pressure reading; a processing device for obtaining a second blood pressure reading by analysis of the pulse of the person obtained by the photoplethysmogram device; configured such that the second blood pressure is capable of being calibrated to the first blood pressure reading before being output by the wearable blood pressure monitor.

Preferably, the blood pressure monitor can be worn on the body for a long period of time for continuous blood pressure monitoring.

Typically, the physical-reaction in the body part is a reaction to a change in the height to which the body part is placed. Preferably, the blood pressure monitor comprises a gravity sensor, gyroscope or a system of accelerometers capable of detecting angular displacement of the body part; such that the height at which the body part is placed is deducible from angular displacement of the body part. Optionally, the body part is the limb of the person. Possibly, the body part may be bicep, wrist, finger or ear canal of the person.

Optionally, the physical-reaction in the body part is the exertion of a pressure by the body part against an external counter-pressure, in which case blood pressure measurement may be obtained by observing the pressure of the bicep, wrist or finger when strangling that bicep, wrist or finger of the person.

In yet a further aspect, the invention proposes a wearable blood pressure monitoring system, comprising: a device for monitoring blood pressure from a first body part of a person using physical-reaction-based measurement; a second device for monitoring blood pressure from a second body part of the person using static measurement; the blood pressure monitoring system configured to calibrate the blood pressure readings from the second device to blood pressure the readings of the first device.

Advantageously, the invention provides the possibility that the entire system can be worn on the body for a long period of time for continuous blood pressure monitoring.

Such a system also provides the advantage that the first device and second device need not be provided as or within a single integral device. The first and second device may be connected by cable or wireless communication so that the blood pressure measurement of one device can be used to monitor the blood pressure measurement of the other device.

Optionally, the first body and the second body part are the same body part. Alternatively, the first body and the second body part are different body parts.

In a further aspect, the invention proposes a method for calibrating a static-observation blood pressure, monitor comprising the steps of: measuring a physical-reaction from a first body part of a person, and interpreting the physical-reaction a calibration blood pressure reading; measuring blood pressure using the static-observation blood pressure monitor from a second body part of the person; and calibrating the blood pressure readings of the static-observation blood pressure monitor using the calibration blood pressure reading.

Optionally, the first body part and the second body part are generally the same body part. Alternatively, the first body part and the second body part are generally different body parts.

Preferably, the physical-reaction is measured with a device which is integral to the static-observation blood pressure monitor.

Preferably, the static-observation blood pressure monitor comprises photoplethysmogram sensor suitable for reading photoplethysmogram to deduce blood pressure.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows a normalized PPG signal useable for the purpose of deducing blood pressure;

FIG. 1a shows a first embodiment of the invention;

FIG. 2 shows a signal obtained by the embodiment of FIG. 1a;

FIG. 3 shows a standard cardiogram for explaining the signal of FIG. 2;

FIG. 4 shows how the embodiment of FIG. 1a is used by a person;

FIG. 5 shows how signals obtain by the embodiment of FIG. 1a;

FIG. 5a shows how a mathematical model fitting may be used in the embodiment of FIG. 5;

FIG. 6 shows how signals obtained, as illustrated in FIG. 5, are applied against a model;

FIG. 7 shows an alternative to the embodiment of FIG. 4;

FIG. 8 is a schematic diagram of the parts within the embodiment of FIG. 1a;

FIG. 9 shows a further alternative to the embodiment of FIG. 4;

FIG. 10 shows a further alternative the model in FIG. 6;

FIG. 11 shows a flowchart of the method employed in the embodiment of FIG. 1a;

FIG. 12 shows another embodiment alternative to that of FIG. 1a;

FIG. 12a shows yet another alternative to the embodiment of FIG. 4;

FIG. 13 shows yet another embodiment alternative to that of FIG. 1a;

FIG. 13a shows how the embodiment of FIG. 1a is used by a person switching between a calibration mode and a regular blood pressure monitoring mode;

FIG. 13b is a flowchart showing how the embodiment of FIG. 1 is used to perform a calibration;

FIG. 13c is a schematic illustration of the first step in calibration, shown in the form on a graph;

FIG. 13d is a schematic illustration of the step in calibration subsequent to that of FIG. 13c;

FIG. 13e is a schematic illustration of the step in calibration subsequent to that of FIG. 13d;

FIG. 14 shows yet another embodiment alternative to that of FIG. 1a;

FIG. 15 shows yet another embodiment alternative to that of FIG. 1a;

FIG. 16 illustrates a part of the embodiment of FIG. 15;

FIG. 17 further illustrates the part shown in FIG. 16;

FIG. 18 illustrates another embodiment alternative to that of FIG. 1a;

FIG. 19 illustrates another embodiment alternative to that of FIG. 1a; and

FIG. 20 illustrates the embodiment of FIG. 19 in use.

### DESCRIPTION OF EMBODIMENTS

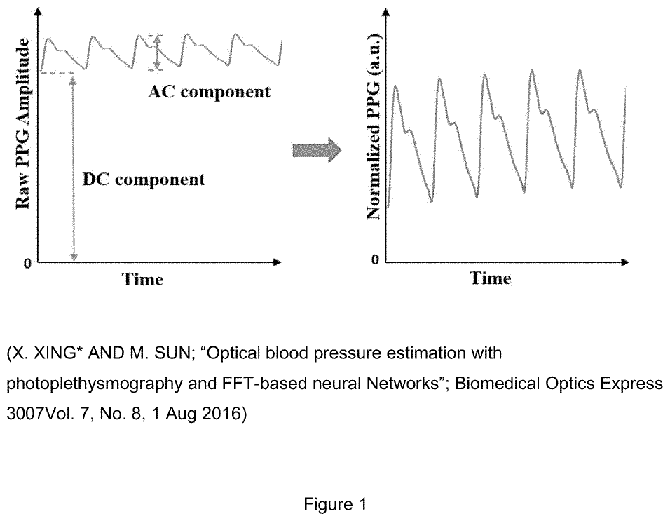

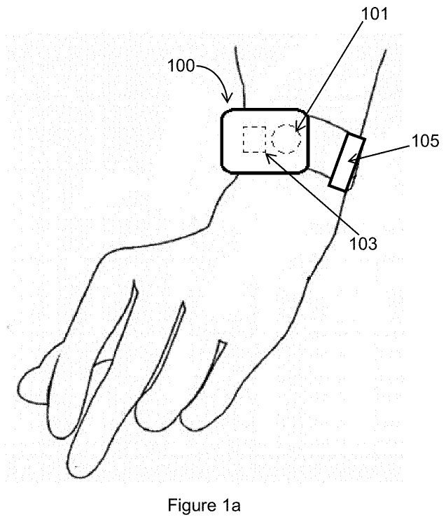

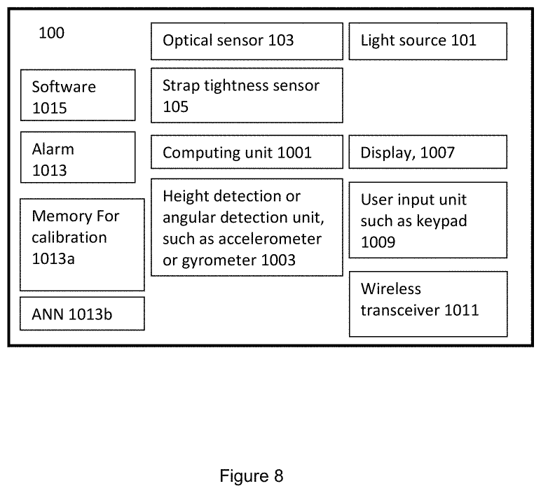

FIG. 1a shows a wrist wearable blood pressure monitor 100 shaped like a watch. The underside of the blood pressure monitor 100 comprises a PPG (photoplethysmocharty) sensor. FIG. 8 is a schematic diagram showing some preferable functional modules which are provided within the blood pressure monitor 100.

The blood pressure monitor 100 has at least two ways of monitoring blood pressure by the same PPG sensor. There are many methods of obtaining blood pressure, and any suitable two ways may be used. However, it is preferable for a First Method that the blood pressure monitor 100 be capable of determining the blood pressure of a person by observing changes in PPG light transmission when the wrist of the person on which the blood pressure monitor is worn is lifted to certain heights. Accordingly, the First Method determines blood pressure from a physical-reaction of the person's body part when he raises his wrist.

It is also preferable, for a Second Method, that the blood pressure monitor 100 be capable of analysing PPG signals obtained by light transmission through the wrist to determine blood pressure, such as the one proposed by Xing et al. The PPG signal analysis may be done mathematically, by a trained artificial intelligence system or by any other analytical methods. Therefore, the Second Method does not measure physical parameters such as height, or pressure around a body part. The Second Method is simply a static method of observation, and monitors the person's blood pressure without the person needing to be actively aware of the Second Method being executed. Accordingly, such static methods are suitable for long term blood pressure monitoring and do not get in the way of the person's daily affairs. In contrast, the person is acutely aware whenever the First Method is being used.

The relation between light transmission to blood pressure varies from person to person, which is due, inter alias, to varying matrix of skin, thickness of tissues and density of blood vessels in the wrist. Therefore, calibration is necessary to interpret PPG signal obtained by the Second Method into accurate blood pressure readings. After the first calibration, it is possible that the light transmission readings may drift in accuracy over time due to use, ambient temperature and casual impacts, and re-calibration using the First Method is required occasionally so maintain the readings of the Second Method accurate.

As the First Method interprets a tangible, measurable physical parameter, such as the height of the wrist, into blood pressure, the blood pressure reading obtained by the First Method is deemed sufficiently accurate. Hence, a blood pressure reading obtained by the First Method may be used to calibrate blood pressure readings obtained by the Second Method. Consequently, this allows the Second Method to be deployed for long term, accurate blood pressure monitoring.

To calibrate the Second Method, the First Method is used to obtain the person's blood pressure reading. This reading of the person's blood pressure is then stored inside a memory 1013a in the blood pressure monitor 100. Subsequently, without taking off the blood pressure monitor 100 or adjusting the position of the blood pressure monitor 100, the blood pressure monitor 100 switches to using the Second Method. As the Second Method commences just a moment after the person's blood pressure has been read by the First Method, the initial PPG signal observed by the Second Method can be equated to the blood pressure reading just obtained by the First Method. In this way, the First Method calibrates the Second Method.

The two methods of monitoring blood pressure are independent techniques, but, in the preferred embodiments, both may be executed by using the same PPG sensor in the blood pressure monitor. As the same parts are used, this provides an advantage that the blood pressure monitor can be made compact and easily wearable for extended periods of time despite employing two different methods of blood pressure monitoring.

1. Detailed Description of the First Method of Obtaining Blood Pressure Used in the Blood Pressure Monitor

A typical PPG sensor comprises at least one light source 101 such as an LED (light emitting diode), which is illustrated by the circle in broken line in FIG. 1a, and at least one corresponding optical sensor 103 normally placed next to the light source 101, which is illustrated by the square in broken line. The broken lines represent the invisibility of the light source 101 and the optical sensor 103 from the side of the blood pressure monitor 100 facing away from the person wearing the blood pressure monitor 100.

The blood pressure monitor 100 has a strap by which it may be worn on the wrist. It is advisable that the PPG sensor is tightly secured against the wrist in order to avoid ambient light from affecting detections of the optical sensor 103. The strap is designed to apply a known, pre-determined pressure around the wrist. The pressure is predetermined by using a pressure sensor 105 provided on the strap. An example of such a pressure sensor 105 is a MEMS (microelectromechanical systems) barometer. The MEMS barometer operates as a mechanical pressure release device, which releases pressure slowly when the strap is overly tightened around the wrist until the pre-determined pressure remains. Alternatively, the pre-determined pressure is provided by using a pre-selected material for the strap, the material having a specific elasticity or resilience which repeatedly applies the same pressure around the wrist every time the blood pressure monitor 100 is worn. Whichever the methods used, the same pressure is applied to the wrist repeatedly every time the same person wears the blood pressure monitor 100.

Light emitted by the light source 101 into the wrist is scattered in all directions by wrist tissue. A portion of the scattered light propagates towards the optical sensor 103. Blood, skin and tissue all absorb a portion of the light. However, the effect of skin and tissue on light propagation is consistent and the extent of light absorbed by skin and tissue does not change noticeably. The amount of blood in the wrist pulsates as the heart pumps. Hence, the amount of light absorbed when the wrist is full of blood is more than the amount of light absorbed when the tissue is relatively depleted of blood.

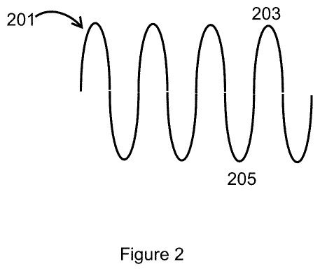

FIG. 2 schematically illustrates the pulsating pattern of light propagation through the person's wrist. The pulsation is cyclical and virtually periodic, corresponding to the heart beat. The peaks 203 in the signal 201 are moments when the heart is relaxed and wrist tissue is relatively depleted of blood, such that more light propagates through the wrist to reach the optical sensor 103. It should be noted that FIG. 2 shows the transmission signal pulse. In an absorption signal pulse the peaks and troughs are inversed.

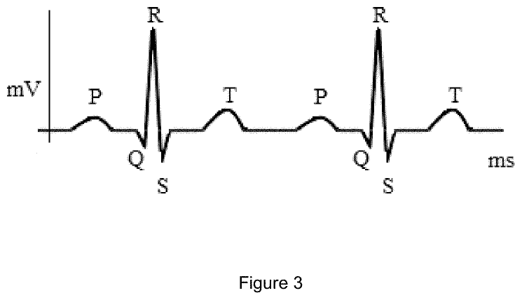

FIG. 3 shows a standard cardiogram having several peaks which can be detected as electric signals from the different heart chambers, represented by PQRST. Each peak 203 of the signal 201 in FIG. 2 corresponds to a period between two adjacent R peaks.

The troughs 205 in the signal 201 in FIG. 2 are moments when the heart contracts and pumps blood into the body, and the wrist tissue is full of blood. A lower amount of light reaches the optical sensor 103 because a significant portion of light from the light source 101 has been absorbed by blood.

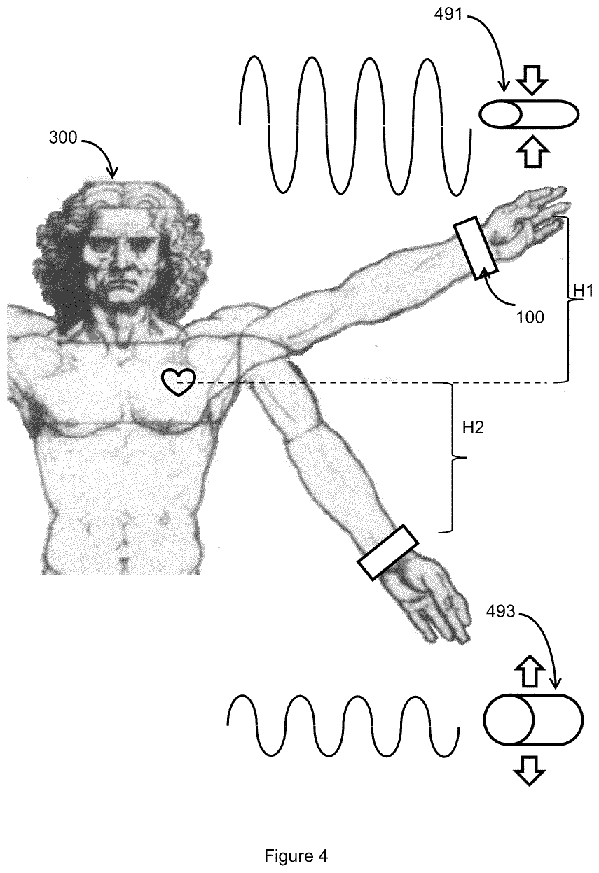

FIG. 4 shows a person 300 wearing the blood pressure monitor 100 of FIG. 1. The strap of the monitor 100 is tied and tightened around his wrist. The consistent pressure applied by the strap acts as a counter-pressure against pressure in blood vessels within the wrist. The counter-pressure causes blood vessels within the wrist to deform slightly. The extent of deformation depends on pressure within the blood vessels and affects the amount of blood which may be pumped into the wrist.

It is well-known that pressure is height-related. There is lower pressure in greater heights above the ground, and there is greater pressure nearer the ground. The same phenomenon can be seen within the wrist. When the person 300 lifts his hand into an elevated position above his heart, blood pressure in his wrist will be lower. A lower blood pressure is relatively weaker against the constant counter-pressure applied by the strap. Therefore, blood vessel deformation due to the counter-pressure from the strap is more pronounced, see label 491 in FIG. 4, and a smaller amount of blood is pumped into blood vessels in the wrist.

Conversely, when the person 300 lowers his hand into a position below his heart, the pressure in blood vessels within the wrist is relatively greater. The greater blood pressure exerting against the counter-pressure allows blood vessels within the wrist to recover a little from their deformation, see label 493 in FIG. 4, and more blood is pumped into blood vessels in the wrist.

Accordingly, FIG. 4 also shows two light propagation signals accompanying the respective blood vessel deformations labelled 491 or 493. The signal at the top of FIG. 4 has greater pulsation amplitudes as the wrist has less blood in the elevated hand position; more light is able to propagate through wrist tissue to reach the optical sensor 103. The signal at the bottom of FIG. 4 has smaller pulsation amplitudes as the wrist has more blood; more blood in the wrist means more of light is absorbed and less light is able to propagate through wrist tissue to reach the optical sensor 103.

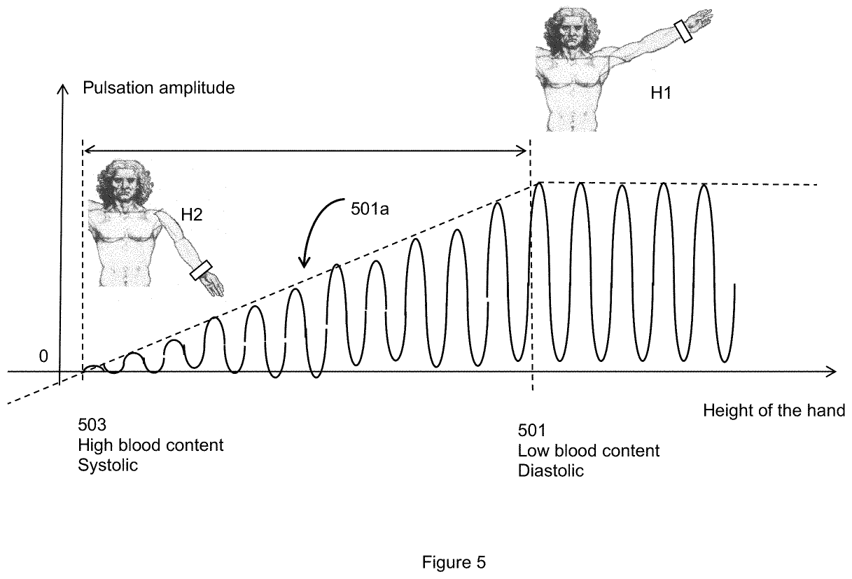

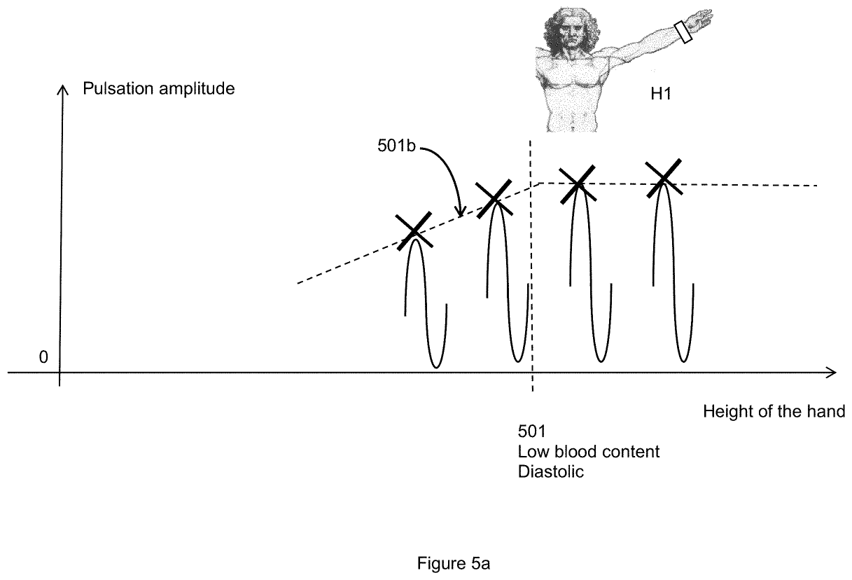

FIG. 5 shows how systolic pressure and diastolic pressure are determined by elevated hand position and lower hand position. The left side of the horizontal axis shows the lower hand position and the right side of the horizontal axis shows the elevated hand position.

To begin monitoring his blood pressure, the person 300 gets himself into a ready position by standing upright and stretching out his hand wearing the pressure monitor 100. The hand is stretched away from his side and lifted to shoulder level. Subsequently, when the person moves his hand down from shoulder level and closes it towards his side, and his wrist eventually moves below the heart. Pressure within blood vessels in the hand increases as he moves his hand downwards and blood pumped into the wrist increases steadily, see the gradient labelled 501a. Thus, the pulsation amplitude of light propagating through wrist tissue reduces as the hand is placed lower. When the hand is lowered to a certain point below the heart, pressure within blood vessels in the wrist is able to overcome the counter-pressure and blood pumped in the wrist is at a maximum amount. At this point, the pulsation amplitude of the light propagating through wrist tissue reaches zero, which is where the gradient of the graph 501a crosses the horizontal axis. The height of the wrist at this crossing as indicated by the dashed vertical line labelled 503 is the height of the wrist at which systolic pressure is manifested. In the unlikely event that the hand has moved and closed against the side of the person 300 but the gradient 501a has not crossed the horizontal axis, the gradient can be extrapolated towards the horizontal axis to do so.

Starting again from shoulder level, the person now moves his out-stretched hand upward. As the hand moves upwards, blood pressure decreases and less blood is pumped in the wrist due to the counter-pressure from the strap. See again gradient labelled 501a. Eventually, the blood pressure is so low that only the diastolic pressure remains in the raised hand. Therefore, when the hand is raised to a certain point above the heart, a steady-state appears: blood pumped into the hand is at a minimum even if the hand is raised further. The diastolic blood pressure provides this minimum blood content in the hand, which translates into minimum light absorption and hence maximum light transmission. The point labelled 501 at which the pulsation of light through the wrist begins to show a steady, maximum amplitude represents the height of the wrist at which diastolic pressure is manifested.

The hand does not need to be raised starting with the wrist below heart level. The hand may be raised starting from shoulder level even though the shoulder is already above the heart. This is because the hand normally needs to be raised well above the shoulder to manifest the diastolic pressure.

The skilled man understands that by ‘steady maximum amplitude’ and ‘steady minimum amplitude’, natural variation of signal amplitudes falling above and below the mean levels may still be observed.

The actual hand positions for manifesting both diastolic pressure and systolic pressure is actually measured from the wrist wearing the blood pressure monitor 100, and the term ‘hand’ is used loosely herein.

It is most easily appreciated if the height of the wrist is measured against the ground. Theoretically, however, the height of the wrist may be referenced from a selected point representing the person's heart position instead. FIG. 4 illustrates this option, showing an elevated wrist height expressed as H1. H1 is the distance between the height of the wrist and the height of the heart that manifests the diastolic pressure, That is, H1 is measured perpendicularly to the ground. Furthermore, FIG. 4 shows a lowered wrist height expressed as H2, where H2 is the distance between the height of the wrist and the height of the heart that manifests the systolic pressure.

The blood pressure monitor 100 described so far may be used to determine diastolic and systolic blood pressures of a person generally, and may be provided to persons as such. However, to evaluate blood pressure more accurately, the amplitude of the light propagating through the wrist may be referenced against a mathematical model.

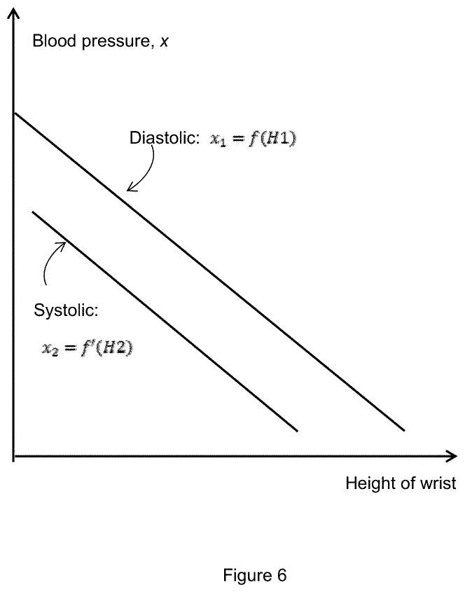

The concept of observing the blood pressure using the First Method is illustrated by a schematic mathematical model in FIG. 6. The horizontal axis of the graph shown in FIG. 6 represents the height of the wrist. The vertical axis represents blood pressure deduced from the height of the wrist. The relationship between the two axes is described by the following equations, (1a) and (1b).

x₁=f(h) (1a)
where
   x₁=diastolic pressure, defined as the minimum value of blood pressure
   h=height of elevation (which is H1 in FIG. 4)

x₂=f′(h) (1b)
where
   x₂=systolic pressure, defined as the maximum value of blood pressure
   h=height of elevation (which is H2 in FIG. 4)

The two functions, f and f′, typically give graphs of similar shapes.

As explained using FIG. 5, H1 is found by the person elevating his hand to discover the point labelled 501 at which light propagation begins to show steady maximum amplitude. H2 is found by the person lowering his hand to discover the point labelled 503 at which the amplitude of light propagation is reduced to zero or crosses the horizontal axis. Accordingly, any change in H1 and H2 for the same person may be attributed to change in his blood pressure, provided that the counter-pressure applied by the strap has been repeated with sufficient precision and accuracy.

In other words, as long as the applied counter pressure is repeatable, the blood pressure monitor 100 can be used to determine the systolic and diastolic blood pressures from the height of the hand.

In practice, the mathematical model can be obtained by proposing a theory or by observing a sampled population of persons wearing the blood pressure monitor 100 and having known systolic and diastolic blood pressures. This relates to statistical methods, which do not require any exposition in this specification.

In some embodiments, the point labelled 501 may be found using a series of discrete positions of the hand, and applying a mathematical model to the discrete positions of the hand, such as fitting a curve thereto. As shown in FIG. 5a, the amplitude of the propagated light is observed when the hand is lifted to four different levels of height, marked by crosses. None of the four different levels of height coincide with the point labelled 501. Nevertheless, the point labelled 501 is found by applying a known mathematical model 501b to the crosses, instead of relying on the blood pressure monitor 100 to detect it actually.

As shown in the schematic diagram in FIG. 8, the blood pressure monitor 100 may comprise a computing module 1001 and suitable software module 1015 for calculating the person's blood pressure from H1 and H2, and a display screen 1007 for displaying the person's systolic and diastolic blood pressures. Alternatively, the blood pressure monitor 100 may rely on a remote device to calculate the systolic and diastolic blood pressures instead of any computing module contained within itself 100. The remote device can be a smart phone which is in wireless communication with the blood pressure monitor 100, and which is able to obtain H1 and H2 from the blood pressure monitor 100 to calculate the person's systolic blood pressure and diastolic blood pressures. In this case, the blood pressure monitor 100 comprises a wireless transceiver 1011 to communicate with a smart phone.

Optionally, an accelerometer or its likes 1003 may be used to determine the exact height that the hand has been raised or lowered to. The use of an accelerometer to do this requires calculating the force as detected to deduce movement and hence, distance moved. This is known to the skilled man and requires no further elaboration here. Of course, an operator may also measures H1 and H2 manually instead, and then enters H1 and H2 into the blood pressure monitor 100 as data input via a keypad 1009 provided on the blood pressure monitor 100.

A memory 1013a is also illustrated in the schematic diagram of FIG. 8, for recording the readings of blood pressure of the person by this First Method, which may then be used later for calibrating the readings of blood pressure of the person by the Second Method.

FIG. 6 illustrates equation (1a) and equation (1b) as linear. However, the linearity is simply an illustration. The case may well be that the equations are non-linear, as a manufacturer producing different embodiments may choose to use. It suffices for the skilled reader to note that actual blood pressure of a person 300 may be expressed as functions of H1 and H2.

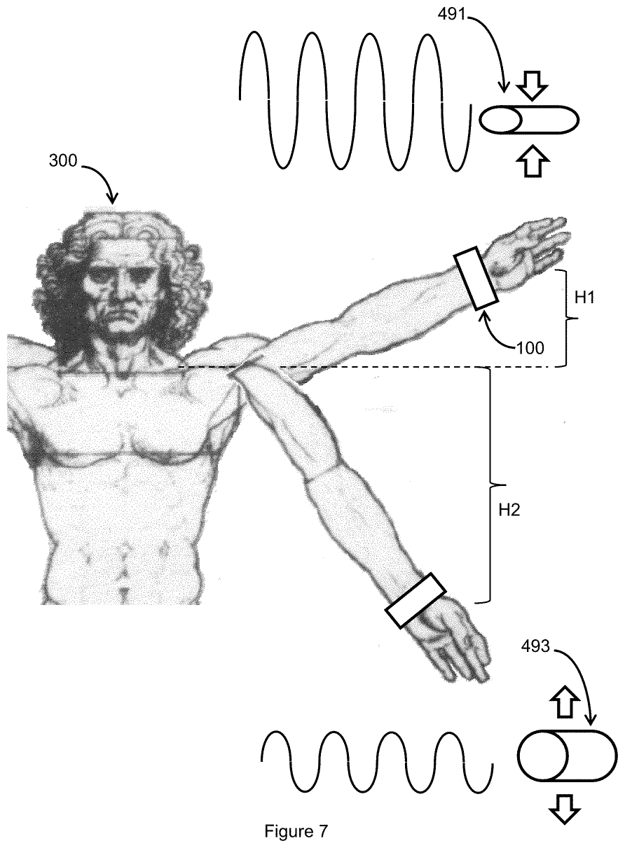

FIG. 7 shows a modification of the embodiment of FIG. 4, wherein H1 and H2 are measured against the shoulder level of the person 300 instead of the ground or from a point representing his heart. This is a more convenient variation of the embodiment as the person's shoulder is more visible than his heart, and the diastolic pressure and systolic pressure normally manifest in wrist positions well above and below the shoulder. The skilled reader will understand that equation (1a) and equation (1b) may be easily adapted to this varied definition of H1 and H2.

One advantage of the described embodiments is that they provide the possibility of using a PPG sensor to observe pulsation of blood (and thereby the blood content in the blood vessels) and then to calculate blood pressure from the pulsation. One of the reason why this can be done lies in the use of a PPG sensor to take different readings from the same body part in different positions. Tissue make-up of the person 300 is the same whether his body part is placed in any position. Therefore, the effects of skin and tissue on propagation of light may be eliminated by taking readings in the different positions, and any change in light propagation between the positions is due to blood content change in the body part.

An advantage of using a distance between two different wrist positions to determine blood pressure is the possibly greater precision which may be had over a mercury column for reading blood pressure. Blood pressure observed by measurement of body fluid, assumed to be equivalent to water, may be more precise due to greater measurement graduation than that of a mercury column. Mercury has a relative density of 13.56 to water, and therefore, any error of 13.56 mm in the wrist position to the shoulder or heart will translate to 1 mm of error in the mercury column. If a manual reading of the mercury column is mis-read by 2 mm, for example, it is the equivalent of mis-reading the position of the wrist by 27.2 mm in the present embodiment, which is an error unlikely to escape notice of any operator of the described embodiments.

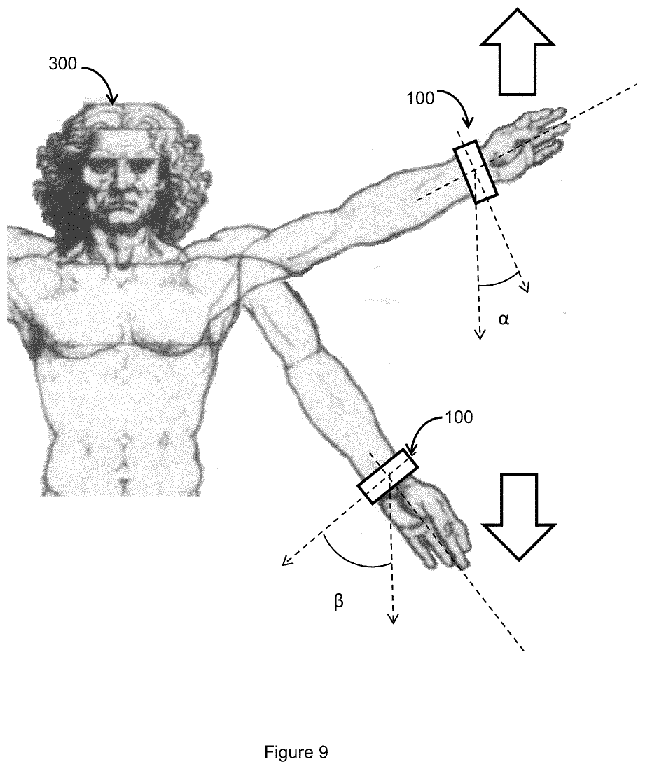

To ensure that H1 and H2 are measured properly, the accelerometer 1003 or any other kind of height detection unit in the blood pressure monitor 100 is used. An alarm 1013 is provided in the blood pressure monitor 100 to issue a suitable calibration alarm if the accelerometer 1003 shows too much deviation at a known height. Nevertheless, it may be difficult to measure H1 and H2, whether they be referenced against the ground, the shoulder or the heart. FIG. 9 shows a more preferable embodiment which overcomes this difficulty, comprising a gyroscope 1003 installed inside the blood pressure monitor 100 to detect angular deviation from the true perpendicular to the ground. The gyroscope 1003 is tuned to be aligned to the true perpendicular pointing to the ground when the wrist wearing the blood pressure monitor 100 is outstretched horizontally. Therefore, when the person wearing the embodiment of FIG. 9 raises his hand from the heart level, the gyroscope is able to detect an angular movement of the blood pressure monitor 100.

When the person stretches his hand outright and raises his hand, at the point where the amplitude of the pulsating light propagating through the wrist reaches a steady maximum, which indicates that blood pulsation in the wrist is at the minimum, a first angle a representing the angular deviation of the blood pressure monitor 100 from the true perpendicular is measured. Accordingly, α is the angle at which diastolic pressure manifests. The skilled reader will understand that α can be used to calculate the extent of upward rotation of the hand or upper limb from the horizontal, about the person's shoulder as the origin.

Conversely, when the person stretches his hand outright and then lowers his hand from his shoulder level, at the point where the amplitude of the pulsating light propagating through the wrist is reduced to zero and crosses the horizontal axis, which indicates that blood pulsation in the wrist is at the maximum, a second angle β representing another angular deviation of the blood pressure monitor 100 to the true perpendicular is measured. β is the angle at which systolic pressure manifests. The skilled reader will understand that β can be used to calculate the extent of downward rotation of the hand or upper limb from the horizontal, about the person's shoulder as the origin.

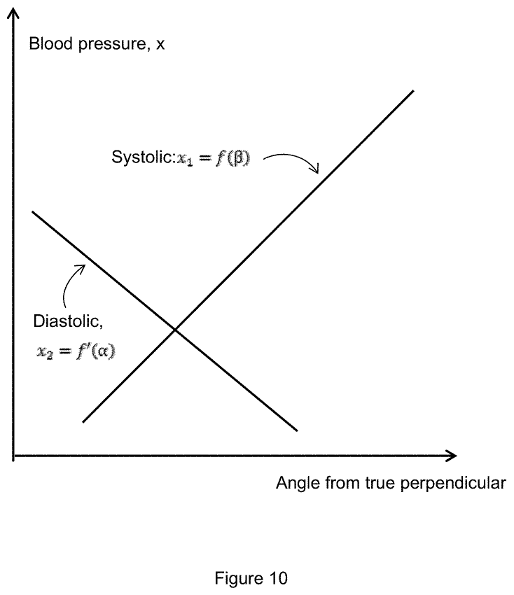

The angles α and β are different for people with different blood pressure levels. As illustrated in the graph of FIG. 10, a person with high diastolic blood pressure has a small α, that is,

$$a ∝ \frac{1}{diastolic blood pressure}$$

The higher the hand needs to be raised above the shoulder to manifest the diastolic pressure, at 501, the greater the angular deviation of a from the true perpendicular, and the lower the person's diastolic blood pressure; the lower need the hand be raised above the shoulder to manifest the diastolic pressure, at 501, the lower the angular deviation of a from the true perpendicular, and the greater the person's diastolic blood pressure.

Conversely, a person with high systolic blood pressure has a greater β, The lower the hand is dropped below the shoulder (and the heart) to manifest the systolic pressure, at 503, the greater the angular deviation of β from the true perpendicular. Accordingly,

β∝systolic blood pressure

In other words, the greater β the greater the systolic blood pressure; a person with low systolic blood pressure needs only to lower his hand slightly to a relatively small β to manifest the systolic blood pressure.

By measuring angular displacement to determine blood pressure, the absolute height of the hand position to the person's heart, shoulder or to the ground does not need to be measured. This is particularly advantageous over the earlier embodiments, as the position on the wrist where the blood pressure monitor 100 is worn may vary easily between different occasions of blood pressure measurement and cause measurements of H1 and H2 to be imprecise. In one application of this embodiment, the person may simply hold on to a door handle and allow himself to stand (so that his hand is below heart level) and squat (so that his hand is above heart level) to determine blood pressure; the gyrometer is able to measure the position of his hand overhead or below the shoulder by angular displacement.

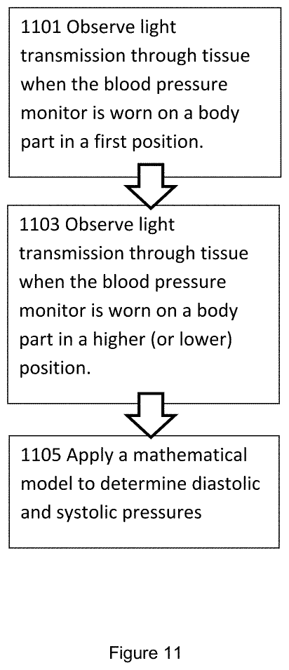

FIG. 11 is a flowchart showing one possible general procedure of determining blood pressure using the embodiment of FIG. 1. At first, in step 1101, light propagation level through a body part, which may be the wrist, is read when the body part is in a first position. This may relate to the hand when it is raised in the afore-described embodiments. Then, in step 1103, light propagation level is read again but when the same part is in a second position, and this may relate to the hand when it is lowered. Of course, the reverse in starting from a hand lowered position and then proceeding to a hand raised position is also within contemplation. The positions of the body part in which the amplitude of light pulsation is at maximum, at 501, and in which amplitude of light pulsation is zero, at 503, are noted and applied against a mathematical model to determine the diastolic blood pressure and the systolic blood pressure, in step 1105.

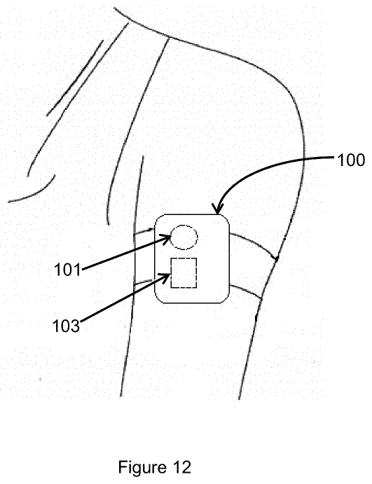

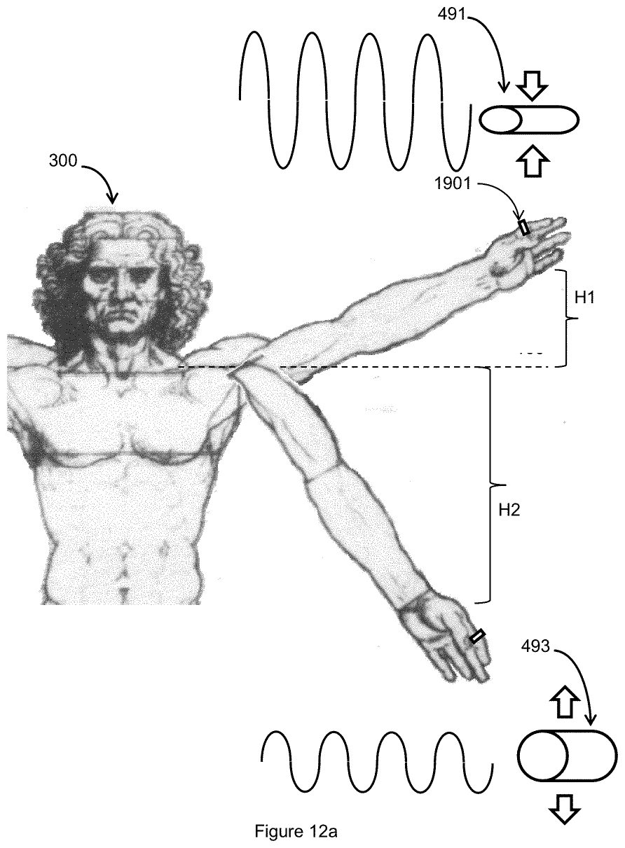

FIG. 12 shows a variation of the embodiment of FIG. 1 a wherein the blood pressure monitor 100 again comprises a PPG sensor, but is worn around the arm or biceps of the person. In order that the blood pressure monitor 100 may be moved across the heart when the arm is raised and lowered, the blood pressure monitor 100 is preferably worn as near to the elbow as possible. Similar to the afore-described examples, H1 and H2 are measured as the distance between the blood pressure monitor 100 and the ground, the person's shoulder or heart level, to calculate blood pressure. Alternatively, the blood pressure monitor 100 may contains a gyrometer for determining angular deviations α and β.

Other parts of limb on the upper body may be used, such as the finger or other parts of the forearm other than the wrist, as long as the part may be lifted above the heart or lowered below the heart.

FIG. 12a is a variation of FIG. 7, showing that the blood pressure monitor 100 can be in the form of a ring 1901, to be worn on a finger of the hand which is moved up and down to different heights to determining the blood pressure.

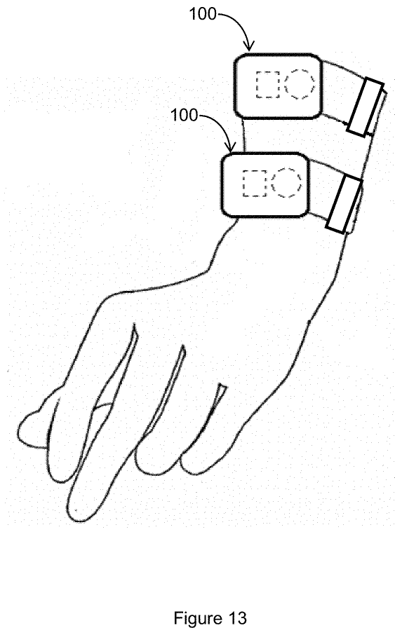

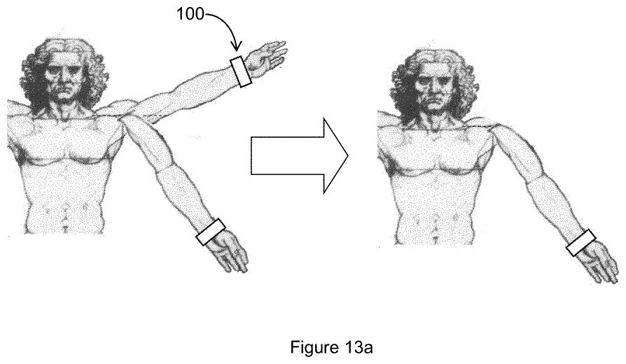

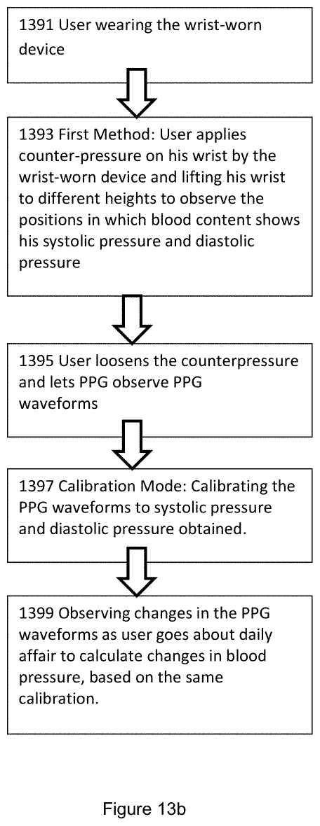

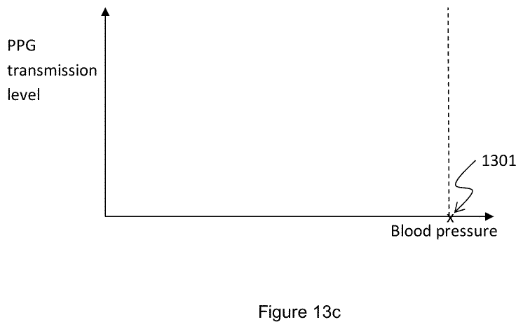

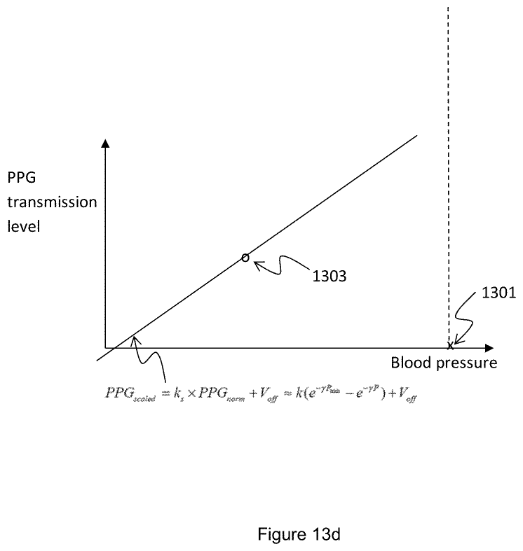

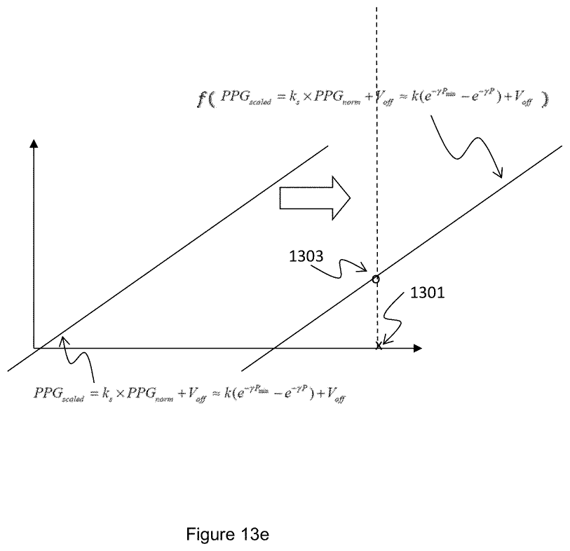

FIG. 13 yet shows another variation of the embodiment which comprises a pair of blood pressure monitors 100, worn in such a way a blood pressure monitor 100 is located on the wrist and another one 100 is adjacent to the first one 100, on the forearm of the same hand away from the wrist. The configuration of the blood pressure monitor 100 to be worn on the forearm may comprise a longer strap and perhaps a stronger light source 101 for illuminating the thicker tissue layers of the forearm, and perhaps a more sensitive optical sensor 103 for detecting light propagation through the thicker forearm. Any inaccuracies or reading discrepancies between the two blood pressure monitors 100 may be mathematically treated and removed, resulting in a more accurate reading of blood pressure. The benefits of the embodiment of FIG. 9 will be more easily appreciated in this embodiment, since the angular displacement from the true perpendicular is the same for both the blood pressure monitors 100 no matter where they are placed along the same limb, whereas H1 and H2 as measured from the heart, shoulder or ground are different for the two blood pressure monitors 100 which thus require a different calculation each. In this embodiment, it is possible to use both the blood pressure monitors on the forearm to obtain the blood pressure of the person according to the First Method, and calibrate only either one of the two blood pressure monitors to obtain and analyse PPG signal for continuous blood pressure monitoring according to the Second Method. Alternatively, it is possible to use either one of the two blood pressure monitors to obtain the blood pressure of the person according to the First Method, and then calibrate both of the two blood pressure monitors to obtain and analyse PPG signal for blood pressure monitoring according to the Second Method.

While embodiments have been described as monitoring the amount of light propagating through the person's skin, blood and tissue, the actual measurement may be taken on either light transmission or light absorption.

Where it has been described that measurement of wrist position starts from shoulder level to move below the shoulder level, the skilled reader understands that the direction of movement is a matter of choice, and the wrist may well start moving from the side of the person towards shoulder level. Similarly, other described limb movements may be executed in the direction which is the reverse of that which has been described.

Although drawings provided in this specification shows a sidewise raising and lowering of the person's hand, it is possible that the hand be raised and lowered when stretched to the front of the person.

The amount of light absorbed by blood in the person's tissue depends on the selected frequency of the light used. Therefore, an optimal frequency is typically selected and used in the embodiments for optimal performance. In some embodiments, however, two or more different light frequencies or frequency ranges are used at once to better discriminate blood pulsation reading from the influence of noise contributing factors such as ambient light and so on. For example, one monochromatic near-infrared frequency and a far infrared frequency are used at the same time.

Accordingly, as one of the simplest embodiments, a method has been described for obtaining the blood pressure of a person 300 comprising steps monitoring pulsation of blood in a body part of the person while moving the body part from a first height to a second height, detecting a first position in which the intensity of the pulsation of blood changes if the body part moves from the first position 501 (or 503) in one direction, and in which the intensity of blood pulsation remains generally constant if the body part moves from the first position 501 (or 503) in the reverse direction, and providing the first position as input to a first calculation model suitable for obtaining the person's blood pressure.

Furthermore, as another one of the simplest embodiments, a blood pressure monitor 100 suitable for being worn on a body part of a person 300 has been described, which comprises a blood pulsation monitor, a movement detector configured to detect a first height position of the body part in which the blood pulsation monitor observes that intensity of pulsation of blood in the body part changes if the body part moves from the first height position 501 (or 503) in one direction, and in which the blood pulsation monitor observes that pulsation of blood remains generally constant if the body part moves from the first height position 501 (or 503) in the reverse direction, and a data treatment module for calculating the person's blood pressure based on the first height position.

2. Detailed Description of the Second Method of Obtaining Blood Pressure Used in the Blood Pressure Monitor

The Second Method of obtaining blood pressure is simply to observe passively the PPG signal obtained by using the same PPG sensor in the blood pressure monitor 100. The preferred method is any analytical method similar to that proposed by Xing et al., without any or with minimum measurements requiring user movements, actions, or applied tension or pressure onto the user. This allows the blood pressure monitor 100 to be used for an extended period of time of static-observation, without interfering with the person's lifestyle; the person is able to wear the blood pressure monitor 100 for extended periods of time like a person would wear a watch without being mindful all the time of himself wearing the blood pressure monitor 100.

To commence monitoring blood pressure using the Second Method, the counter-pressure applied by the strap for the First Method is relieved somewhat. This makes it comfortable enough for the person to wear the blood pressure monitor 100 for a long period of time. The same PPG sensor in the blood pressure monitor then commences to observe PPG signal by emitting light into the wrist and detecting light transmission through the person tissue. The PPG signal observed is subject to mathematical signal analysis to determine blood pressure, as described by Xing et al.

However, at the commencement of the Second Method, the PPG sensor operates in a Calibration Mode first. In the Calibration mode, both the systolic and diastolic blood pressures read by the First Method (stored in the memory 1013a inside the blood pressure monitor 100) are used to evaluate the PPG signal as read and analysed by the Second Method. Typically, as it was just a moment ago when the First Method was used to observe the person's blood pressure, it is unlikely that the person's blood pressure has changed much. Hence, the blood pressure as read by the Second Method may be compared with the blood pressure as read by the First Method to obtain a correction factor by which all subsequent blood pressure readings using the Second Method are adjusted, or calibrated.

The way in which the systolic and diastolic blood pressures can be observed from the PPG signal, in order to be calibrated against the systolic and diastolic blood pressures observed by the First Method, may include using the trained artificial neural network trained to do so. An artificial neural network module 1013b is illustrated in the schematic diagram of components in the blood pressure monitor 100 shown in the drawing of FIG. 8. There are many researches in such an area and it is not necessary to elaborate herein how an artificial neural network can be trained to observe the two different types of blood pressures from the PPG signal. Furthermore, there are method which measures blood pressure by monitoring the pulse transit time, which relies on using an electrocardiogram and a photoplethysmogram to detect a pulse. The time difference between the pulse as detected by the electrocardiogram and the photoplethysmogram has been used in some analytical methods to deduce blood pressure. All such methods, i.e. methods based on signal analysis, tends to drift and a method of performing re-calibration easily, such as that provided by the First Method, makes it possible for the person or consumer to correct the drift at any time by himself.

Other methods of measuring pulse transit time include measuring the time difference between electrocardiogram and ballistocardiograms, which can then be used for deducing blood pressure monitoring. Such a method also needs to be calibrated, and can be done by asking the person monitored to wear an embodiment like that of FIG. 1 and use the embodiment as described.

3. Detailed Description of the Calibration Procedure

FIG. 13a and the flowchart of FIG. 13b show how the blood pressure monitor 100 is used during a Calibration Mode.

Before the Calibration Mode begins, the person first wears the blood pressure monitor 100 on his wrist, at step 1391. Then the counter-pressure is applied to tighten the blood pressure monitor around the person's wrist, in step 1393. In FIG. 13a, the drawing shows on the left side how a person may lift his hand up and down to obtain his systolic and diastolic blood pressure using the First Method, at step 1395, which is calculated from the height positions of the wrist. After the First Method is completed, the person loosens the counter-pressure, step 1395.

Typically, the PPG sensor may take about one to five minutes for the First Method to obtain a PPG signal stable enough for deducing the person's blood pressure for use in calibration of the Second Method. The extent of “stability” may be pre-determined by the manufacturer of the embodiment, and is usually a standard deviation of PG signal readings within a certain limit. This is production-specific and does not require elaboration here.

The blood pressure monitor 100 enters the Calibration Mode, at step 1397, for calibrating the Second Method. The person simply stays somewhat still for the PPG sensor to take a stabilised reading of the PPG signal. The ratio between the blood pressure reading deduced by the Second Method and the blood pressure reading read by the First Methods is used calculate the correction factor to be applied to all blood pressure readings of the Second Method henceforth, thereby completing the Calibration Mode.

Subsequently, person carries on with his daily activities, at step 1399. The blood pressure monitor 100 remains worn on his wrist. If the signal as read by the PPG sensors changes, it means his blood pressure has changed. It is possible for a person's blood pressure to vary throughout the day, such as when he has exercised or subjected himself to strong emotions. Hence, the Calibration Mode should be conducted right after the blood pressure (both the systolic and diastolic) has been obtained by the First Method for better calibration, as there is unlikely to be any change in the blood pressure so quickly.

If the blood pressure monitor 100 is the type shown in FIG. 12 which is secured to the biceps, the operation is the same as with the wrist worn type. The counter-pressure around the bicep is released first, and the blood pressure monitor enters into the Calibration mode.

In a variation of the embodiments, a historical record of the correction factor is kept to provide an averaged correction factor (or a moving-averaged correction factor). Over time, however, the average correction factor will be more and more accurate by eliminating randomness or fluctuation in the correction factor. The historical record of the correction factor can also be used to train the artificial neural network 1013b in the blood pressure monitor 100 to adjust or calibrate readings obtained by the Second Method.

FIG. 13c is a schematic illustration of the calibration, shown in the form on a graph. Using the First Method, the blood pressure of the person is read, as is indicated as x in the horizontal axis of the graph.

Subsequently, as shown in FIG. 13d, using the Second Method, the person's blood pressure is read again. The Second Method interprets the PPG transmission into a blood pressure reading, indicated as o 1303, based on a linear model such as Equation (1) which correlates blood pressure to PPG transmission. However, the blood pressure interpreted Equation (1) is different from the blood pressure measured by the First Method; the blood pressure deduced using the Second Method is too low, as compared to the blood pressure read by the First Method.

Hence, the blood pressure monitor calculates the difference between two blood pressure readings, i.e. o 1303 and x 1301, and provides a correction factor. Graphically, as illustrated in FIG. 13e for this example, this is the same as moving the linear model towards the right of the graph, until the blood pressure point o 1303, 1303, of the curve is moved to the same place as x 1301 along to the horizontal axis. Mathematically, this may be described as a function of Equation (1), as shown below.

f(PPG_{scaled}=kₛ×PPGₙₒᵣₘ+V_{off}≈k(e^{-γP}^{ₘᵢₙ}−e^{-γP})+V_{off})

Subsequently, any PPG readings would be interpreted using the adjusted curve or formula. The adjustment is the correction factor.

Optionally, the mathematical treatment of the adjustment can be polynomial or in any way deemed the best model in the opinion of the specific manufacturer of the embodiment, and does not need specific elaboration here.

The above embodiments are described as using the same PPG sensor in both the First Method and Second Method. In some embodiments, there may be two PPG sensors provided within the same integral blood pressure monitor, with one being dedicated to apply the First Method while the second being dedicated to apply the Second Method.

Furthermore, in some embodiments, it is possible that a light frequency more suitable for monitoring blood content in the body part is used in the First Method but another frequency which is more suitable for obtaining analysable PPG signal is used in the Second Method. The frequency used for the Second Method may be selected to avoid noise from surrounding infrared emissions due to body heat, for example, since the

Second Method is expected to be used for a long period of time to observer the person's blood pressure.

4. Further Variations of the Embodiments

Although embodiments using the First Method have been described with a person 300 wearing a blood pressure monitor 100 in an upright position, and moving his hand up and down vertically, it is possible in other embodiments for the person to be wearing the blood pressure monitor 100 lying on a bed (not illustrated). In such embodiments, the person moves his hand wearing the blood pressure monitor 100 from his back to his front in the transverse plane instead of the coronal plane, and therefore also moving his hand passed his heart vertically.

In yet a further embodiment using the First Method, the embodiment is ankle-worn instead of wrist-worn. The person simply lies down on a bed to monitor his blood pressure. One reading is taken when the leg wearing the embodiment is rested on the bed and levelled with the heart, and another when the leg is lifted up into the air and raised above the heart, in order to measure the systolic and diastolic blood pressure.

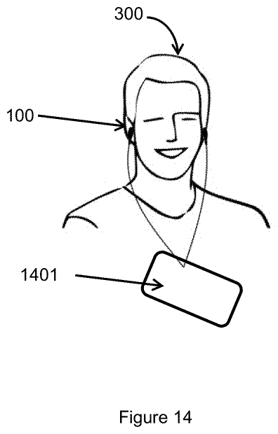

In yet another embodiment, a blood pressure monitor 100 can be provided in the form of an ear worn device, as illustrated in FIG. 14. This ear worn device comprises an ear bud which is installed with a light source 101 and an optical sensor 103, as well as a gyrometer. The ear bud is shaped to be suitable for being inserted into the ear canal.

The blood pressure monitor 100 has either wired or wireless connection to a device such as a display screen or smart phone 1401 which can display the readings of the blood pressure monitor 100. The display screen or the smart phone 1401 also provides the person 300 with interface for controlling the operation of the blood pressure monitor 100. Furthermore, a smart phone may provide the processing and memory resource for calculating blood pressure based on the readings observed by the optical sensor 103 in the ear bud 1501. This allows the ear bud 1501 to be small, as a processor is not strictly needed to be installed into the ear bud 1501. In contrast, the embodiment of FIG. 1a is large enough to contain its own processor and memory, and therefore the embodiments like that may be easily operated without need of an accompanying smart phone. The requirement of a suitable application in the smart phone 1401 for operating the blood pressure monitor 100 is a well-known concept and does not require any exposition in this specification.

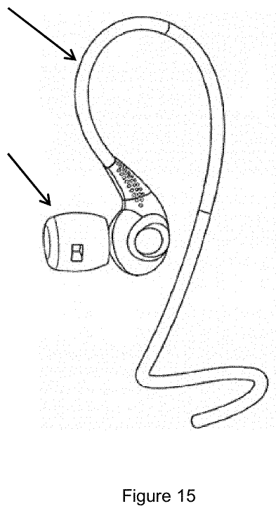

Optionally, it may be the sole function of the blood pressure monitor 100 of FIG. 14 to measure the blood pressure of the person 300 wearing it. Alternatively, however, the ear bud type blood pressure monitor 100 is also an ear phone, or a hearing aid. FIG. 15 shows this variation of the embodiment of FIG. 14 as a hook-on earphone, the speaker of which is encased in an ear bud 1501 meant for insertion into the ear canal. As with the embodiment of FIG. 14, the ear bud 1501 is installed with a light source 101 and sensor for monitoring blood pulsation in the ear canal.

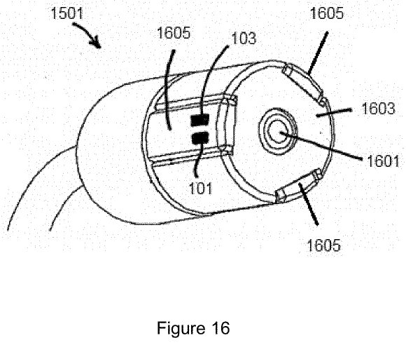

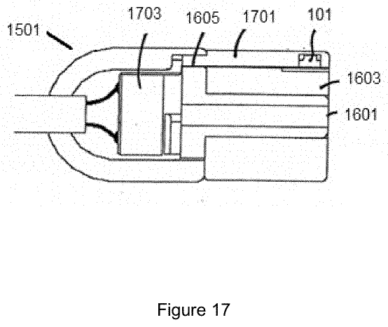

FIG. 16 and FIG. 17 show how a light source 101 and an optical sensor 103 may be arranged in the ear bud 1501 of the embodiment of FIG. 15. Typically, the ear bud 1501 is made of a deformable resilient outer part 1701 sized to fit within the ear canal of a person.

FIG. 16 illustrates the core parts of the ear bud 1501 with the resilient outer part removed. FIG. 17 shows the resilient outer part 1701 assembled into the embodiment.

Within the ear bud 1501 is a speaker 1703, a hollow inner core 1601 for sound conduction from the speaker in to the ear, an resilient inner foam structure 1603 for softness and flexibility, thin wirings (not illustrated) for connection to the light source 101 and optical sensor 103. The resilient outer part 1701 provides increased comfort and protection of the light source 101 and optical sensors 103. The resilient inner foam 1603 may be compressed during insertion of the ear bud 1501 into the ear to provide further support in the ear canal. FIG. 16 illustrates three sets 1605 of light source 101 and optical sensor 103 pairs, spaced 120 degree around the ear bud. One set is facing the reader showing clearly the light source 101 and optical sensor 103, while the other sets face away from the reader.

To obtain the blood pressure of the person 300 wearing the embodiment 100 of FIG. 14 or FIG. 15 using the First Method, the person 300 lies down to take a first reading of light propagation through his ear tissue, and then sits up to take another reading of light propagation through his ear tissue. In other words, change in blood pulsation in the ear canal between the positions of lying down and sitting up is noted by observing an accompanying change in the amplitude of light propagation through ear tissue. When the person is lying down, the ear is just about level with the heart and has a similar effect on blood pressure within the ear canal as if the ear canal is below heart level. When the person sits up, the ear is well above the heart. Therefore, this embodiment also comprises a gyroscope to detect the lying down and sitting up of the person 300 and to obtain a and for calculating the systolic pressure and diastolic blood pressure, or to obtain the height of the ear canal when the person 300 sits up to determine H1 to calculate diastolic pressure. In this embodiment, there is no counter-pressure applied against wall of the ear canal.

In a variation, the person wearing the ear bud 1501 can have his systolic or diastolic blood pressures measured by squatting down and standing up, to allow the PPG sensor in the ear bud 1501 observe any change in blood content in the ear canal, which may be used to deduce the blood pressures.

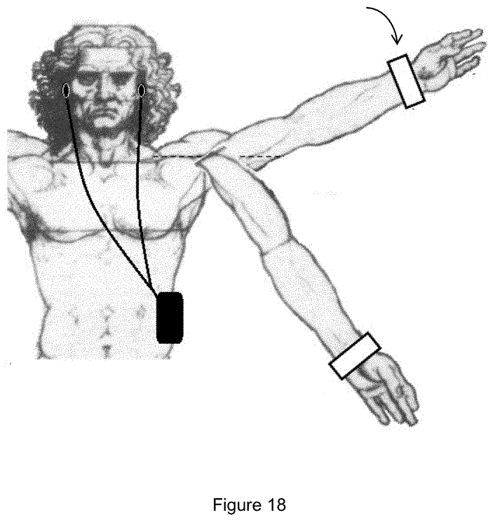

FIG. 18 shows yet another embodiment. In this case, the person wears a blood pressure monitor 100 on his wrist for obtaining blood pressure of the person by using the First Method, i.e. by means of height displacement of a body part, such as the wrist. However, another blood pressure monitor in the form of an earbud can be used to monitor continuously the person's blood pressure by the Second Method. In other words, the blood pressure monitor in the form of an earbud is a different device to that of the blood pressure monitor 100 on the wrist.

Both the earbud-type blood pressure monitor and the blood pressure monitor 100 on the wrist are in wireless communication with the smartphone shown carried by the person (or any other computing device). Hence, the smartphone is able to manage the use of the two devices, and let one calibrate the other.

Specifically, the earbud-type blood pressure monitor can enter into a Calibration Mode once the blood pressure monitor 100 on the wrist has obtained the person's blood pressure by observing height displacement of the body part using the First Method. In other words, in some embodiments, the device operating the First Method does not need to be the device operating the Second Method but the device operating the First Method can still be used to calibrate the readings of the device operating the Second Method.

This embodiment allows any existing PPG signal based blood pressure monitoring device to be re-calibrated by a physical parameter based blood pressure reading. Only the software of these existing devices needs to be upgraded to be capable of operating in the Calibration Mode in order to obtain the correction factor.

Although amplitude of light propagation is used in the afore-mentioned embodiments to monitor blood content using the First Method, it is possible that non-optical measurement is used instead as the First Method. For example, the blood pressure monitor 100 may comprise a tonometer in place of the PPG, or the blood pressure monitor may comprise a digital sphygmomanometer which requires strangling a body part such as the bicep to obtain the systolic and diastolic blood pressures). A tonometer is an instrument for measuring the pressure in a part of the body or a blood vessel. The amplitude of the pulsation can be correlated to the blood pressure in a similar way as the pulsating amplitude of propagated light. The skilled reader will note that a greater amplitude in the afore-described embodiments using a PPG sensor refers to greater light signal and hence less blood in the wrist whereas, if a tonometer is used, a greater amplitude represent more blood pumped into the wrist. Both calibration and equations as discussed can be adapted accordingly.

In yet further embodiments in which a PPG sensor is installed in a earbud device such as an ear phone, It is also possible that a tonometer is used in the ear canal instead of the light source 101 and optical sensor 103.

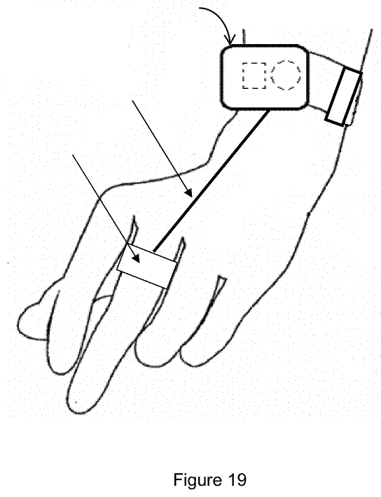

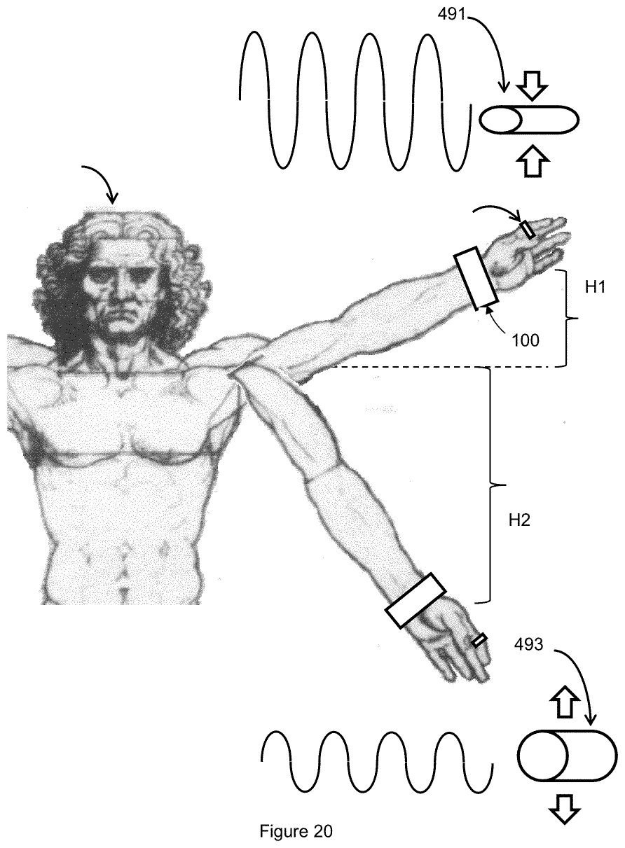

FIG. 19 shows yet a further variation of the embodiments, in which a wrist based blood pressure monitor 100 is used together with another blood pressure monitor 1901 which is in the form of a ring that can be worn on a finger. A PPG sensor is provided inside the ring (not illustrated) for monitoring blood content. The wrist based blood pressure monitor 100 and the finger based blood pressure monitor 1901 are shown linked by a communication cable. Optionally, the communication between the wrist based blood pressure monitor 100 and the finger based blood pressure monitor 1901 can be wireless (not illustrated). FIG. 20 shows how the embodiment of FIG. 19 may be used to obtain blood pressure readings using the First Method. Either the finger based blood pressure monitor 1901 or the wrist based blood pressure monitor 100 may be used to obtain the blood pressure readings for used in the Calibration Mode, to calibrate the other one of the finger based blood pressure monitor 1901 or the wrist based blood pressure monitor 100.

Accordingly, embodiments have been described some of which comprises a method for calibrating a blood pressure monitor 100 comprising steps of: providing the blood pressure monitor 100, the blood pressure monitor 100 being capable of measuring blood pressure in two different ways; and using the first way of measuring blood pressure to calibrate the second way of measuring blood pressure.

Accordingly, embodiments have been described some of which comprises a wearable blood pressure monitor 100, comprising: a photoplethysmogram device for observing blood content in a body part of a person wearing the blood pressure monitor 100; a measurement device for measuring physical-reaction in the body part; a processing device for interpreting the physical-reaction in the body part into a first blood pressure reading; a processing device for obtaining a second blood pressure reading by analysis of the pulse of the person obtained by the photoplethysmogram device; configured such that the second blood pressure is capable of being calibrated to the first blood pressure reading before being output by the wearable blood pressure monitor 100.

Accordingly, embodiments have been described some of which comprises a wearable blood pressure monitor 100ing system, comprising: a device for monitoring blood pressure from a first body part of a person using physical-reaction-based measurement; a second device for monitoring blood pressure from a second body part of the person using static measurement; the blood pressure monitor 100ing system configured to calibrate the blood pressure readings from the second device to blood pressure the readings of the first device.

Accordingly, embodiments have been described some of which comprises a method for calibrating a static-observation blood pressure, monitor comprising the steps of: measuring a physical-reaction from a first body part of a person, and interpreting the physical-reaction a calibration blood pressure reading; measuring blood pressure using the static-observation blood pressure monitor 100 from a second body part of the person; and calibrating the blood pressure readings of the static-observation blood pressure monitor 100 using the calibration blood pressure reading.

While there has been described in the foregoing description preferred embodiments of the present invention, it will be understood by those skilled in the technology concerned that many variations or modifications in details of design, construction or operation may be made without departing from the scope of the present invention as claimed.

Although a human has been described as the subject for blood pressure monitoring, the embodiments may be adapted to monitor the blood pressure of any animal which is capable of wearing a device configured to detect blood pulsation, and wherein the body part is capable of moving between two positions relative to the animal's heart.

1. A method for calibrating a blood pressure monitor comprising steps of:
providing the blood pressure monitor, the blood pressure monitor being capable of measuring blood pressure in two different ways; and
using the first way of measuring blood pressure to calibrate the second way of measuring blood pressure.

2. A method for calibrating a blood pressure monitor as claimed in claim 1, wherein
the first way obtains blood pressure by a physical-reaction-based measurement of the person wearing the blood pressure monitor.

3. A method for calibrating a blood pressure monitor as claimed in claim 1, wherein
the second way obtains blood pressure by at least one static measurement of the pulse of the person wearing the blood pressure monitor.

4. A method for calibrating a blood pressure monitor as claimed in claim 3, wherein the static measurement comprises the step of:
transmitting light through a body part of the person to monitor blood content.

5. A method for calibrating a blood pressure monitor as claimed in claim 2, wherein the physical-reaction-based measurement comprises the step of:
measuring the height to which a body part is lifted which manifests an accompanying change in blood content in the body part.

6. A method for calibrating a blood pressure monitor as claimed in claim 2, wherein the physical-reaction-based measurement comprises the steps of:
strangling the body part with a known counter-pressure; and
measuring the pressure exerted by a body part against the counter-pressure.

7. A method for calibrating a blood pressure monitor as claimed in claim 6, wherein
the body part is a finger of the subject; and
the strangling is provided by a ring worn on the finger.

8. A method for calibrating a blood pressure monitor as claimed in claim 4, wherein
the measuring in the first way and the blood content monitoring in the second way are performed by using a same photoplethysmogram sensor on the same body part.

9. A method for calibrating a blood pressure monitor as claimed in claim 8, wherein
the body part is the ear canal.

10. A method for calibrating a blood pressure monitor as claimed in claim 4, wherein
the measuring in the first way and the blood content monitoring in the second way are performed by using different photoplethysmogram sensors on different body parts respectively.

11. A wearable blood pressure monitor, comprising:
a photoplethysmogram device for observing blood content in a body part of a person wearing the blood pressure monitor;
a measurement device for measuring physical-reaction in the body part;
a processing device for interpreting the physical-reaction in the body part into a first blood pressure reading;
a processing device for obtaining a second blood pressure reading by analysis of the pulse of the person obtained by the photoplethysmogram device;
configured such that the second blood pressure is capable of being calibrated to the first blood pressure reading before being output by the wearable blood pressure monitor.

12. A wearable blood pressure monitor as claimed in claim 11, wherein
the physical-reaction in the body part is a reaction to a change in the height to which the body part is placed.

13. A wearable blood pressure monitor as claimed in claim 12, wherein
the blood pressure monitor comprises a gravity sensor capable of detecting angular displacement of the body part; such that
the height at which the body part is placed is deducible from angular displacement of the body part.

14. A wearable blood pressure monitor as claimed in claim 12, wherein:
the body part is the limb of the person.

15. A wearable blood pressure monitor as claimed in claim 14, wherein:
the body part is a finger of the person.

16. A wearable blood pressure monitor as claimed in claim 12, wherein:
the body part is the ear canal of the person.

17. A wearable blood pressure monitor as claimed in claim 11, wherein
the physical-reaction in the body part is the exertion of a pressure by the body part against an external counter-pressure.

18. A wearable blood pressure monitoring system, comprising:
a device for monitoring blood pressure from a first body part of a person using physical-reaction-based measurement;
a second device for monitoring blood pressure from a second body part of the person using static measurement;
the blood pressure monitoring system configured to calibrate the blood pressure readings from the second device to blood pressure the readings of the first device.

19. A wearable blood pressure monitoring system as claimed in claim 18 wherein,
the first body and the second body part are the same body part.

20. A wearable blood pressure monitoring system as claimed in claim 18 wherein,
the first body and the second body part are different body parts.

21. A method for calibrating a static-observation blood pressure, monitor comprising the steps of:
measuring a physical-reaction from a first body part of a person, and interpreting the physical-reaction a calibration blood pressure reading;
measuring blood pressure using the static-observation blood pressure monitor from a second body part of the person; and
calibrating the blood pressure readings of the static-observation blood pressure monitor using the calibration blood pressure reading.

22. A method for calibrating a static-observation blood pressure monitor as claimed in claim 21, wherein
the first body part and the second body part are generally the same body part.

23. A method for calibrating a static-observation blood pressure monitor as claimed in claim 22, wherein
the physical-reaction is measured with a device which is integral to the static-observation blood pressure monitor.

24. A method for calibrating a static-observation blood pressure monitor as claimed in claim 21, wherein
the first body part and the second body part are generally different body parts.

25. A method for calibrating a static-observation blood pressure monitor as claimed in claim 21, wherein
the static-observation blood pressure monitor comprises photoplethysmogram sensor suitable for reading photoplethysmogram to deduce blood pressure.

26.-28. (canceled)
